(19) United States
(12) Patent Application Publication
(10) Pub. No.: US 2025/0179519 A1
(43) Pub. Date: Jun. 5, 2025

(54) PROCESS TO ENHANCE VIRAL VECTOR PRODUCTION IN CELL-CULTURE USING GLYCYL-GLUTAMINE

(71) Applicant: Evonik Operations GmbH, Essen (DE)

(72) Inventors: Johanna Peters, Marl (DE); Tilman Sauter, Meerbusch (DE); Martin Schilling, Bonn (DE)

(73) Assignee: Evonik Operations GmbH, Essen (DE)

(21) Appl. No.: 18/842,083

(22) PCT Filed: Feb. 28, 2023
(86) PCT No.: PCT/EP2023/054954

§ 371 Date: Aug. 28, 2024

(30) Foreign Application Priority Data
Mar. 3, 2022 (EP) 22159915.2

(51) Int. Cl. C12N 15/86; C07K 5/062

(52) U.S. Cl. CPC C12N 15/86; C07K 5/06026; C12N 2750/14143; C12N 2750/14151

(57) ABSTRACT

A method manufactures an RNA- or DNA-containing virus particle in cell culture, a supplement for a culture medium and a culture medium for producing of RNA or DNA-containing virus particles having one or more dipeptides or derivatives thereof, wherein one dipeptide is glycyl-glutamine (Gly-Gln), by providing a cell capable of producing the virus particle, contacting the cell with a culture medium with at least one dipeptide or derivatives thereof and obtaining the virus particle from the culture medium or from the cell.

### FIELD OF THE INVENTION

The present invention relates to dipeptides and their uses in cell culture. Moreover, the present invention relates to biotechnological production processes. More specifically, the present invention relates to a process to enhance viral vector production in cell-culture using glycyl-glutamine.

### BACKGROUND OF THE INVENTION

Viral vectors are increasingly used for therapeutic applications. Prominent examples approved for marketing authorization in the European and/or US market include vaccine applications such as the viral vector-based Ebola vaccine (ERVEBO®, based on vesicular stomatitis virus [1]), COVID-19 vaccines (Vazxevria™, Ad26.COV2.S, based on adenovirus (Ad) [2,3]) or gene therapies (Luxturna®, Zolgensma®, based on Adeno-associated virus (AAV) 2 resp. AAV9 [4, 5]). Additionally, various clinical trials based on viral vectors are ongoing with over 200 approved clinical trials alone for adeno-associated viruses [6]. As biopharmaceutical development progresses from clinical into commercial phases, an increasing demand for efficient viral vector production processes is seen.

Viral vectors are produced through a cell culture process. A liquid medium is required to support cell growth, transfection and ultimately viral vector production. While the use of serum and serum-derived (mainly fetal bovine serum or FBS) proteins is still common in academic settings and small-scale clinical trials, industrial production in stirred-tank bioreactors is mainly serum-free to limit supply chain, regulatory and quality control risks. A key challenge remains to replace serum without losing performance in terms of cell density and viability as well as productivity [7]. Current research focuses on a better understanding of individual media components while trying to replace animal and human derived serum to increase reproducibility in each production batch and to reduce blood-derived components in therapeutic manufacturing processes [8].

Glutamine is a component of particular interest in mammalian cell culture media. For optimal growth of cells, recommended glutamine levels are 3-10 times higher compared to other amino acids in the medium [9]. As it is poorly soluble, unstable at elevated temperatures (e.g., upon heat sterilization) and thereby forming cytotoxic pyroglutamate and ammonia, it must be added directly prior to use. In contrast, glutamine-dipeptides such as alanyl-glutamine or glycyl-glutamine were found to not only offer enhanced solubility (568 and 154 g/L at 20° C. in water for Aln-Gln and Gly-Gln compared to 36 g/L for Gln) but also to be comparably stable during autoclaving and storage. As a result, they can entertain high growth rates indicated by short doubling times even after sterilization [11]. An approach to further enhance the heat stability of glutamine-dipeptides included the n-acylation of such dipeptides resulting in further improved heat stability while maintaining positive effects on cell growth similar to the corresponding non-acylated dipeptide [12].

State-of-the art animal component-free and chemically defined media formulations optimized for propagation and transfection of resp. viral vector production in HEK 293 cells are still formulated without Gln to avoid the formation of degradation products. They require addition of either Gln or glutamine dipeptides prior to use. Manufacturer issued standard recommendations for media use typically include addition of Gln or Ala-Gln to a final concentration of 4 mM to 8 mM prior to use [13, 14].

While the standard recommendations include Ala-Gln, the specific difference between Gly-Gln and Ala-Gln has not been investigated so far in viral vector production. Christie et al. have shown that high cell yields can be obtained in antibody-secreting mouse hybridoma (CC9C10) culture containing either of Ala-Gln or Gly-Gln but monoclonal antibody production was comparable in all three cultures independent of glutamine source (Gln, Ala-Gln, Gly-Gln). This was still valid despite the even higher concentration of Gly-Gln (20 mM) compared to Ala-Gln (6 mM) employed in the study. Neither HEK cell lines nor viral vector production nor animal-origin free media were part of the evaluation.

A similar observation is described in WO2011133902A2 [16], which describes a method to manufacture nucleic acid containing virus particles in animal cell culture including different dipeptides. In this document, literature on glutamine peptides as cited previously is mentioned but it focuses on the use of cysteine- and tyrosine-dipeptides. The patent application referenced further just discloses that Gln is unstable and can be replaced with Ala-Gln or Gly-Gln but does not describe an increase in viral vector titers.

In contrast, WO2019195729A1 focuses on AAV production, it discloses that Ala-Gln is employed and leads to at least equivalent full/empty ratios (determined based on TEM or analytical ultracentrifugation) but does not describe a similar effect for Gly-Gln. Despite having employed HEK cells, the study also employed DMEM as exemplary medium instead of an animal-origin free medium as described in the present study, As a result, there is no indication to be found that the change of Gln source (e.g., from Ala-Gln to Gly-Gln) positively influences the viral vector productivity or quality in HEK cell-based production processes. While cell culture media described in the prior art containing glutamine, glutamine-dipeptides or derivatives of glutamine-dipeptides suffice to enhance cell proliferation and thereby cell-culture based processes solely depending on cell growth and resulting density, there is still a need for media or media supplements enhancing product quality, e.g., by improving the yield of a certain component of interest compared to its by-products.

Critical quality attributes (CQA) related to strength and potency of viral vector production include the number of viral genomes (vg) and capsids as well as the full/empty-ratio calculated based on vg/capsid titer ratio or other methods. The vg titer, typically assessed by quantitative polymerase chain reaction (qPCR), is commonly used to control dosing as it is directly related to the therapeutic effect [18]. The capsid or viral particles (vp) titer, typically assessed by serotype-specific enzyme-linked immunosorbent assay (ELISA), measures viral particles such as intact virions and empty capsids. The determination of both titers as well as the ratio is recommended for gene therapy products by European Medicines Agency (EMA) as well as US Food and Drug Administration (US FDA) [20]. Depending on the product's control strategy, empty capsid particles are removed and result in more complex downstream purification or remain in the final product. There, they can reduce transduction efficiency by competing with fully packaged vector particles for binding with/uptake by target cells and may cause dose-limiting side effects based on immunogenicity through capsid-specific CD8+ T cell response thereby negatively influencing therapeutic success [22]. Downstream purification to eliminate empty capsids is associated to challenges such as poor scalability in case of ultracentrifugation or variable efficiency depending on vector/serotype and added complexity combined with industrial waste from column resin regeneration in case of ion exchange chromatography [23].

### SUMMARY OF THE INVENTION

The above shortcomings are addressed by the present invention. The invention is defined by the terms of the appended independent claims. Preferred embodiments of the invention are defined by the dependent claims.

A lab study to evaluate the influence of glutamine-dipeptides on viral vector production (AAV8) was conducted in HEK293 cells. The evaluation of transfection efficiency by fluorescence measurement of green-fluorescent protein-positive cells, the capsid titer in the supernatant determined by ELISA as well as the genomic titer determined from supernatant by qPCR surprisingly indicated more efficient transfection as well as increased genomic and capsid titer for the cultures supplemented with glycyl-glutamine (Glycyl-L-Glutamine hydrate, Gly-Gln) compared to the other variants. Compared to alanyl-glutamine supplementation, which resulted in a higher capsid titer while featuring genomic titers comparable to the other processes, the high genomic titer is remarkable indicating more efficient production and addressing the challenges related to empty capsids/the full/empty-ratio described above. Compared to state-of-the-art glutamine supplementation, which contributes to higher yields based on promoting cell growth, a direct influence on the production phase independent of the cell number was seen.

Therefore, the invention relates to a method of manufacturing an RNA or DNA-containing virus particle in cell culture comprising the steps of
providing a cell capable of producing said virus particle;
contacting said cell with a culture medium comprising one or more dipeptides or derivatives thereof, wherein one dipeptide is glycyl-glutamine (Gly-Gln); and
obtaining said virus particle product from said culture medium or from said cell.

Another aspect of the invention relates to a supplement for a culture medium for use in the production of RNA or DNA-containing virus particles, comprising one or more dipeptides or derivatives thereof, wherein one dipeptide is glycyl-glutamine (Gly-Gln) and a culture medium for use in the production of RNA or DNA-containing virus particles, comprising one or more dipeptides or derivatives thereof, wherein one dipeptide is glycyl-glutamine (Gly-Gln).

The invention further relates to the use of the dipeptide glycyl-glutamine (Gly-Gln) or a derivative thereof for the production of virus particles in cell culture.

Preferred embodiments of the invention are described in further detail in the following detailed description of the invention.

### DETAILED DESCRIPTION OF THE INVENTION

A “peptide” shall be understood as being a molecule comprising at least two amino acids covalently coupled to each other by alpha-peptide bonds (R¹—CO—NH—R²).

A “dipeptide” shall be understood as being a molecule comprising two amino acids covalently coupled to each other by a peptide-bond (R¹—CO—NH—R²) it may also be present as a salt or in hydrate form.

An “amino acid”, in the context of the present invention, shall be understood as being a molecule comprising an amino functional group (—NH₂) and a carboxylic acid functional group (—COOH), along with a side-chain specific to the respective amino acid. In the context of the present invention, both alpha- and beta-amino acids are included. Preferred amino acids of the invention are alpha-amino acids, in particular the 20 “natural amino” acids including cystine as follows:
Alanine (Ala/A)
Arginine (Arg/R)
Asparagine (Asn/N)
Aspartic acid (Asp/D)
Cysteine (Cys/C)
Cystine (Cyss/C2)
Glutamic acid (Glu/E)
Glutamine (Gln/Q)
Glycine (Gly/G)
Histidine (His/H)
Isoleucine (Ile/I)
Leucine (Leu/L)
Lysine (Lys/K)
Methionine (Met/M)
Phenylalanine (Phe/F)
Proline (Pro/P)
Serine (Ser/S)
Threonine (Thr/T)
Tryptophan (Trp/W)
Tyrosine (Tyr/Y)
Valine (Val/V)

In the context of the present invention, the expression “natural amino acids” shall be understood to include both the L-form and the D-form of the above listed 20 amino acids. The L-form, however, is preferred. In one embodiment, the term “amino acid” also includes analogues or derivatives of those amino acids.

A “free amino acid”, according to the invention (for instance “free cysteine”), is understood as being an amino acid having its amino and its (alpha-) carboxylic functional group in free form, i.e., not covalently bound to other molecules, e.g., an amino acid not forming a peptide bond. Free amino acids may also be present as salts or in hydrate form. When referring to an amino acid as a part of, or in, a dipeptide, this shall be understood as referring to that part of the respective dipeptide structure derived from the respective amino acid, according to the known mechanisms of biochemistry and peptide biosynthesis.

A “growth factor”, according to the invention, shall be understood as being any naturally occurring substance capable of stimulating cellular growth, proliferation and cellular differentiation. Preferred growth factors are in form of protein or steroid hormone. According to one embodiment of the invention, the expression “growth factor” shall be interpreted as relating to a growth factor selected from the list consisting of fibroblast growth factor (FGF), including acidic FGF and basic FGF, insulin, insulin-like growth factor (IGF), epithelial growth factor (EGF), nerve growth factor (NGF), platelet-derived growth factor (PDGF), and transforming growth factor (TGF), including TGFalpha and TGFbeta, cytokine, such as interleukins 1, 2, 6, granulocyte stimulating factor, and leukocyte inhibitory factor (LIF).

An “oligopeptide”, according to the invention, shall be understood as being a peptide compound consisting of 2 to 20 amino acids. More preferred oligopeptides of the inventions are oligopeptides consisting of 2-10 amino acids, 2-6 amino acids, 2-5 amino acids, 2-4 amino acids, or 2-3 amino acids. Most preferred oligopeptides according to the invention are dipeptides.

A “culture medium”, according to the invention, shall be understood as being a liquid or solid medium containing nutrients, the medium being suitable for nourishing and supporting life and/or product formation of cells in the culture. The cultured cells, according to the invention, may be bacterial cells, yeast cells, fungal cells, animal cells, such as mammalian cells or insect cells, and/or plant cells, e.g., algae. Typically, a culture medium provides essential and non-essential amino acids, vitamins, at least one energy source, lipids, and trace elements, all required by the cell for sustaining life, growth and/or product formation. The culture medium may also contain components that enhance growth and/or survival above the minimal rate, including hormones and growth factors. The culture medium has preferably a pH and a salt concentration which supports life, growth and/or product formation of the cells. A culture medium, according to the invention, preferably comprises all nutrients necessary to sustain life and proliferation of the cell culture. Preferred culture media are defined media.

A “chemically defined medium”, according to the invention is a medium that contains no cell extracts, cell hydrolysates, or protein hydrolysates. Chemically defined media comprise no components of unknown composition. As is commonly understood by the person skilled in the art, chemically defined media are usually free of animal-derived components. All components of a chemically defined medium have a known chemical structure. Culture media other than defined culture media may be referred to as “complex” culture media.

A “cell culture medium” shall be understood as being a culture medium suitable for sustaining life, proliferation and/or product formation of animal cells and/or plant cells.

A “basal medium” or “basal culture medium” shall be understood as being a solution or substance containing nutrients in which a culture of cells is initiated.

A “feed medium” shall be understood as being a solution or substance with which the cells are fed after the start of the cultivation process. In certain embodiments, a feed medium contains one or more components not present in a basal medium. The feed medium can also lack one or more components present in a basal medium. Preferably, the concentration of nutrients in the feed medium exceeds the concentration in the basal medium to avoid a loss of productivity by dilution.

A “perfusion medium” shall be understood as being a solution or substance containing nutrients that is continuously added after the beginning of a cell culture, in which harvest is continuously removed.

A “cell culture supplement” shall be understood as being an additive to a culture medium, chemically defined medium or cell culture medium beneficial to enhance healthy expansion, productivity or specific application of cells. One of such applications can be the improvement of cell growth, viability and productivity under serum-free or low serum conditions. It may contain dipeptides, amino acids, glucose, vitamins and proteins.

Examples of derivatives of Gly-Gln may include N-acylated dipeptides, they may include N-acetyl-glycyl-L-glutamine, N-formyl-glycyl-L-glutamine, N-propionyl-glycyl-L-glutamine, N-succinyl-glycyl-L-glutamine, N-acyl esters of the dipeptide or salts of the respective structures.

The expression “N-acylated”, with reference to a chemical compound, such as an amino acid, shall be understood as meaning that the N-acylated compound is modified by the addition of an acyl group to a nitrogen functional group of said compound. Preferably, the acyl group is added to the alpha-amino group of the amino acid.

Surprisingly, it was found that genomic and capsid titer in HEK-293-cell-based viral vector production (AAV8) were substantially increased upon glycyl-l-glutamine supplementation.

Therefore, the present invention relates to a method of manufacturing an RNA- or DNA-containing virus particle in cell culture comprising the steps of
providing a cell capable of producing said virus particle;
contacting said cell with a culture medium comprising one or more dipeptides or derivatives thereof, wherein one dipeptide is glycyl-glutamine (Gly-Gln); and
obtaining said virus particle product from said culture medium or from said cell.

In the context of the present invention, a “cell capable of producing said virus particles” shall include cells that do contain integrated helper viral nucleic acid sequences and those that do not contain integrated helper viral nucleic acid sequences, which achieve packaging of the (recombinant) viral genome reliant on transfecting an additional nucleic acid construct. It shall also include all cells that need any combination of helper viruses, transfection reagents or plasmids to produce virus particles.

As viruses naturally introduce their genetic material into target cells as part of their replications cycle, they can be employed to deliver their RNA or DNA into a host cell for replication. Engineered RNA- or DNA-containing virus particles or viral vectors harness this ability to deliver the genetic material of interest into a target cell. In some of the embodiments, this includes introduction of a therapeutic gene into target cells, leading to expression of a transgene for therapeutic purpose. To date, several viruses have been employed to form engineered RNA- or DNA-containing virus particles for therapeutic applications.

The expression “RNA- or DNA-containing virus particles” should be understood as engineered virus particles featuring the ability to deliver genetic material into a target cell. More specifically, they should be understood as a composition of a capsid and genetic material.

The “capsid” can either be an enveloped or nonenveloped, protein shell of 20-100 nm in diameter that surrounds an inner RNA- or DNA-containing core. In the context of the present invention, specifically AAV capsids of approx. 22 nm in diameter, Ad capsids of 70-100 nm in diameter and lentiviral (LV) capsids of 80-100 nm in diameter are included. The capsid can have different special affinities for certain host cell receptors based on the serotype.

The “genetic material” can either be double-stranded DNA, single-stranded DNA or single-stranded RNA. In a preferred embodiment, it consists of linear, double-stranded DNA ranging from up to 40 kb in length or linear, single-stranded DNA of up to 5 kb in length or single stranded RNA of up to 10 kb in length.

In one embodiment of the invention, the method of culturing cells comprises contacting the cell with a basal culture medium under conditions supporting the cultivation of the cell and supplementing the basal cell culture medium with a concentrated medium according to the present invention. In preferred embodiments, the basal culture medium is supplemented with the concentrated feed or medium on more than one day.

Empty capsids compete with full capsids for binding sites on target cells and hence reduce the therapeutically desired delivery of DNA into them, hence being considered as product-related impurities. These impurities should be measured and may be reported as a ratio, for example, full/empty ratio or virus particles/infectious units ratio. Both can be considered as a critical quality attribute (CQA) in therapies based on virus particles.

In a preferred embodiment, the loading of virus particles with nucleic acids is at least 5% full/empty ratio (ratio of genomic titer with relation to capsid titer), preferably at least 10%, more preferably at least 20%.

In a preferred embodiment, the “full/empty ratio” shall be defined as viral genome titer (vg) divided by viral particle titer (vp). A suitable method to determine vg is quantitative polymerase chain reaction (qPCR). A suitable method to measure vp titer including intact virions and empty capsids is serotype-specific enzyme-linked immunosorbent assay (ELISA).

Alternative analytical methods to determine viral particle and/or viral genome titer or directly derive a full/empty ratio may be based on anion-exchange high-performance liquid chromatography (AEX-HPLC), absorbance ratios at wavelengths of 260/280 nm, cryoelectron microscopy (cryoEM), size-exclusion chromatography multiangle light scattering (SEC-MALS), charge detection mass spectrometry (CDMS), sedimentation velocity analytical ultra centrifugation (SV-AUC) among others. Other names for said “full/empty ratio” such as packaging ratio or capsid titer-to-genome titer ratio or ratios that can be calculated thereof might be used alternatively.

In a preferred embodiment, the virus particles are from Adenoviruses, Lentiviruses or Adeno-associated viruses (AAV), preferably AAV8 or AAV2.

In the context of the present invention, virus particles include those generated of adenoviruses (Ad), adeno-associated viruses (AAV), lentiviruses (LV) herpes simplex viruses and vaccinia viruses. In case of AAV, they are composed of a protein shell surrounding a single-stranded DNA genome of up to approx. 5 kilobases (kb), in case of LV they are composed of a protein shell containing two copies of single-stranded RNA genome of up to approx. 10 kb and in case of Ad, they are composed of a protein shell surrounding double-stranded DNA genome of up to approx. 40 kb. The virus particles can be employed to administer recombinant RNA or DNA into target cells. More than 50 serotypes of Ad and more than eight serotypes of AAV have been identified. These serotypes exhibit a diverse range of tropisms and immune response profiles leading to different applications of interest. The efficiency and specificity of AAV gene delivery can be improved using point mutations on the viral capsid [26, 27].

In a preferred embodiment, the virus particles are from Adenoviruses (Ad), Lentiviruses (LV) or Adeno-associated viruses (AAV), preferably AAV8 or AAV2; however, also virus particles comprising point-mutated AAV8 and AAV2 capsids and chimeras resulting from the transfer of larger peptide domains from or into AAV8 and AAV2 a serotype to another are included.

Cultivation of cells, according to the invention can be performed in batch culture, in fed-batch culture or in continuous culture.

In a preferred embodiment, the dipeptide is present in the culture medium at a final concentration of from 0.1 mM to 20 mM, or 0.1 mM to 10 mM, or 0.5 mM to 10 mM, or 1 mM to 10 mM, or 5 mM to 10 mM.

In a preferred embodiment, the culture medium further comprises at least one carbohydrate, at least one free amino acid, at least one inorganic salt, a buffering agent and/or at least one vitamin.

In a preferred embodiment, said cells are selected from the list consisting of CHO cells, COS cells, VERO cells, BHK cells, HEK cells, HELA cells, AE-1 cells, insect cells, Sf9 cells, TT-D6 cells, BLKCL.4 primary skin fibroblasts, A549 human adenocarcinoma cells, or fibroblast cells, muscle cells, nerve cells, stem cells, skin cells, endothelial cells, immune cells such as NK or T-cells and hybridoma cells, preferably HEK cells.

Another aspect of the present invention relates to a supplement for a culture medium for use in the production of RNA or DNA-containing virus particles, comprising one or more dipeptides or derivatives thereof, wherein one dipeptide is glycyl-glutamine (Gly-Gln).

In a preferred embodiment, the supplement further comprises at least one carbohydrate, at least one free amino acid, at least one inorganic salt, a buffering agent and/or at least one vitamin.

A further aspect of the present invention relates to a culture medium for use in the production of RNA or DNA-containing virus particles, comprising one or more dipeptides or derivatives thereof, wherein one dipeptide is glycyl-glutamine (Gly-Gln).

In a preferred embodiment, the supplement is in liquid form, in form of a gel, a powder, a granulate, a pellet or in form of a tablet.

In a preferred embodiment, the dipeptide is present in the culture medium at a concentration of from 0.1 mM to 20 mM, or 0.1 mM to 10 mM, or 0.5 mM to 10 mM, or 1 mM to 10 mM, or 5 mM to 10 mM.

In a preferred embodiment, said culture medium is in liquid form, in form of a gel, a powder, a granulate, a pellet or in form of a tablet.

In a preferred embodiment, the culture medium is as an aqueous stock or feed solution.

In preferred embodiments, the dipeptide is not N-acylated. N-acylation is known to improve heat stability of certain dipeptide; however, it has been found that N-acylated dipeptides may also lead to inferior viable cell density and viability.

In a specific configuration, the present invention is directed to a cell or tissue culture medium comprising the composition according to the present invention, which further comprises at least one carbohydrate, at least one free amino acid, at least one inorganic salt, a buffering agent and/or at least one vitamin. In a particularly preferred embodiment, the culture medium comprises all of at least one carbohydrate, at least one free amino acid, at least one inorganic salt, a buffering agent and at least one vitamin.

In one embodiment of the invention, the culture medium does not contain growth factors. In accordance with this embodiment, the dipeptide of the invention may be used instead of a growth factor for promoting growth and/or proliferation of the cells in culture. In another embodiment of the invention, the culture medium does not contain any lipids.

In preferred embodiments, the culture medium of the invention is a defined medium, or a serum-free medium. For example, the compositions of the intervention may be supplemented to the Freestyle™ F17 medium, the Freestyle™ 293 medium, the Expi293™ medium all of Gibco™ ThermoFisher (Waltham, USA), the TheraPEAK™ SfAAV™ Medium of LONZA (Basel, Switzerland), the HEK ViP NB of Xell (Bielefeld, Germany), HyClone™ SFM4HEK293 of Cytiva (Marlborough, USA). The dipeptides of the invention may also be supplemented to DMEM medium (Life Technologies Corp., Carlsbad, USA). The invention, however, is not limited to supplementation of the above media.

In other preferred embodiments, the culture medium is a liquid medium in 2-fold, 3-fold, 3.33-fold, 4-fold, 5-fold or 10-fold concentrated form (volume/volume), relative to the concentration of said medium in use. This allows preparation of a “ready-to-use” culture medium by simple dilution of the concentrated medium with the respective volume of sterile water. Such concentrated forms of the medium of the invention may also be used by addition of the same to a culture, e.g., in a fed-batch cultivation or perfusion process.

The cell culture medium (cell or tissue culture basal, feed or perfusion medium) of the present invention may preferably contain all nutrients required for sustained growth and product formation. Recipes for preparing culture media, in particular cell culture media, are well known to the person skilled in the art (see, e.g., Cell Culture Technology for Pharmaceutical and Cell-Based Therapies, Öztürk and Wei-Shou Hu eds., Taylor and Francis Group 2006) [24]. Various culture media are commercially available from various sources.

The culture media of the invention may preferably include a carbohydrate source. The main carbohydrate used in cell culture media is glucose, routinely supplemented at 5 to 25 mM. In addition, any hexose, such as galactose, fructose, or mannose or a combination may be used.

The culture medium typically may also include at least the essential amino acids (i.e., His, Ile, Leu, Lys, Met, Phe, Thr, Try, Val) as well as non-essential amino acids. A non-essential amino acid is typically included in the cell culture medium if the cell line is not capable of synthesizing the amino acid or if the cell line cannot produce sufficient quantities of the amino acid to support maximal growth. In addition, mammalian cells can also use glutamine as a major energy source. Glutamine is often included at higher concentrations than other amino acids (2-8 mM). However, as noted above, glutamine can spontaneously break down to form ammonia and certain cell lines produce ammonia faster, which is toxic.

The culture media of the invention may preferably comprise salts. Salts are added to the cell culture medium to maintain isotonic conditions and prevent osmotic imbalances. The osmolality of a culture medium of the invention is about 300 mOsm/kg, although many cell lines can tolerate an approximately 10 percent variation of this value or higher. The osmolality of some insect cell cultures tends to be higher than 300 mOsm/kg, and this may be 0.5 percent, 1 percent, 2 to 5 percent, 5-10 percent, 10-15 percent, 15-20 percent, 20-25 percent, 25-30 percent higher than 300 mOsm/kg. The most commonly used salts in cell culture medium include Na⁺, K⁺, Mg²⁺, Ca²⁺, Cl⁻, SO₄²⁻, PO₄³⁻, and HCO₃⁻ (e.g., CaCl₂), KCl, NaCl, NaHCO₃, Na₂HPO₄).

Other inorganic elements may be present in the culture medium. They include Mn, Cu, Zn, Mo, Va, Se, Fe, Ca, Mg, Si, and Ni. Many of these elements are involved in enzymatic activity. They may be provided in the form of salts such as CaCl₂), Fe(NO₃)₃, MgCl₂, MgSO₄, MnCl₂, NaCl, NaHCO₃, Na₂HPO₄, and ions of the trace elements, such as, selenium, vanadium and zinc. These inorganic salts and trace elements may be obtained commercially, for example from Sigma (Saint Louis, Missouri).

The culture media of the invention preferably comprise vitamins. Vitamins are typically used by cells as cofactors. The vitamin requirements of each cell line vary greatly, although generally extra vitamins are needed if the cell culture medium contains little or no serum or if the cells are grown at high density. Exemplary vitamins preferably present in culture media of the invention include biotin, choline chloride, folic acid, i-inositol, nicotinamide, D-Ca⁺⁺-pantothenate, pyridoxal, riboflavin, thiamine, pyridoxine, niacinamide, A, B₆, B₁₂, C, D₃, E, K, and p-aminobenzoic acid (PABA).

Culture media of the invention may also comprise serum. Serum is the supernatant of clotted blood. Serum components include attachment factors, micronutrients (e.g., trace elements), growth factors (e.g., hormones, proteases), and protective elements (e.g., antitoxins, antioxidants, antiproteases). Serum is available from a variety of animal sources including human, bovine or equine serum. When included in cell culture medium according to the invention, serum is typically added at a concentration of 5-10% (vol.). Preferred cell culture media are serum-free.

To promote cell growth in the absence of serum or in serum reduced media, one or more of the following polypeptides can be added to a cell culture medium of the invention.

In other embodiments, the cell culture medium does not comprise polypeptides.

One or more lipids can also be added to a cell culture medium of the invention, such as linoleic acid, linolenic acid, arachidonic acid, palmitoleic acid, oleic acid, polyenoic acid, and/or fatty acids of 12, 14, 16, 18, 20, 22, or 24 carbon atoms (each carbon atom branched or unbranched), phospholipids, lecithin (phosphatidylcholine), and cholesterol. One or more of these lipids can be included as supplements in serum-free media. Phosphatidic acid and lysophosphatidic acid stimulate the growth of certain anchorage-dependent cells, such as MDCK, mouse epithelial, and other kidney cell lines, while phosphatidylcholine, phosphatidylethanolamine, and phosphatidylinositol stimulate the growth of human fibroblasts in serum-free media. Ethanolamine and cholesterol have also been shown to promote the growth of certain cell lines. In certain embodiment, the cell culture medium does not contain a lipid.

One or more carrier proteins, such as bovine serum albumin (BSA) or transferrin, can also be added to the cell culture medium. Carrier proteins can help in the transport of certain nutrients or trace elements. BSA is typically used as a carrier of lipids, such as linoleic and oleic acids, which are insoluble in aqueous solution. In addition, BSA can also serve as a carrier for certain metals, such as Fe, Cu, and Ni. In protein-free formulations, non-animal derived substitutes for BSA, such as cyclodextrin, can be used as lipid carriers.

One or more attachment proteins, such as fibronectin, laminin, and pronectin, can also be added to a cell culture medium to help promote the attachment of anchorage-dependent cells to a substrate.

The cell culture medium can optionally include one or more buffering agents. Suitable buffering agents include, but are not limited to, N-[2-hydroxyethyl]-piperazine-N′-[2-ethanesulfonic acid] (HEPES), MOPS, MES, phosphate, bicarbonate and other buffering agents suitable for use in cell culture applications. A suitable buffering agent is one that provides buffering capacity without substantial cytotoxicity to the cells cultured. The selection of suitable buffering agents is within the ambit of ordinary skill in the art of cell culture.

Polyanionic or polycationic compounds may be added to the culture medium to prevent the cells from clumping and to promote growth of the cells in suspension.

In a preferred embodiment, the culture medium is in liquid form. The culture medium, however, can also be a solid medium, such as a gel-like medium, e.g. an agar-agar-, carrageen- or gelatine-containing medium (powders, aggregated powders, instantized powders etc.). Preferably, the culture medium is in sterile form.

The culture medium of the present invention can be in concentrated form. It may be, e.g., in 2- to 100-fold concentrated form, preferably in 2-fold, 3-fold, 3.33-fold, 4-fold, 5-fold, 10-fold, 20-fold, 50-fold or 100-fold (relative to a concentration that supports growth and product formation of the cells). Such concentrated culture media are helpful for preparing the culture medium for use by dilution of the concentrated culture medium with an aqueous solvent, such as water. Such concentrated culture media may be used in batch culture but are also advantageously used in fed-batch or continuous cultures, in which a concentrated nutrient composition is added to an ongoing cultivation of cells, e.g., to replenish nutrients consumed by the cells during culture.

In other embodiments of the invention, the culture medium is in dry form, e.g., in form of a dry powder, or in form of granules, or in form of pellets, or in form of tablets.

The present invention also relates to the use of a culture medium of the invention for culturing cells. Another aspect of the invention relates to the use of a culture medium of the invention for producing a cell culture product.

Another aspect of the present invention relates to the use of the dipeptide glycyl-glutamine (Gly-Gln) or a derivative thereof for the production of virus particles in cell culture. This dipeptide can be used as a supplement in specific cell culture media used for the production of virus particles in cell culture, preferably for the production of virus particles are from Adenoviruses (Ad), Lentiviruses (LV) or Adeno-associated viruses (AAV), preferably AAV8 or AAV2.

### LITERATURE

1: https://www.ema.europa.eu/en/documents/assessment-report/ervebo-epar-public-assessment-report_en.pdf
2: https://www.ema.europa.eu/en/documents/assessment-report/vaxzevria-previously-covid-19-vaccine-astrazeneca-epar-public-assessment-report_en.pdf
3: https://www.ema.europa.eu/en/documents/assessment-report/covid-19-vaccine-janssen-epar-public-assessment-report_en.pdf
4: https://www.ema.europa.eu/en/documents/assessment-report/luxturna-epar-public-assessment-report_en.pdf
5: https://www.ema.europa.eu/en/documents/assessment-report/zolgensma-epar-public-assessment-report_en.pdf
6: Kuzmin, D. A., Shutova, M. V., Johnston, N. R., Smith, O. P., Fedorin, V. V., Kukushkin, Y. S., van der Loo, J., & Johnstone, E. C. (2021). The clinical landscape for AAV gene therapies. Nature reviews. Drug discovery, 20(3), 173-174. https://doi.org/10.1038/d41573-021-00017-7 https://media.nature.com/original/magazine-assets/d41573-021-00017-7/18790666
7: Masri, F., Cheeseman, E., & Ansorge, S. (2019). Viral vector manufacturing: how to address current and future demands. Cell Gene Ther. Insights, 5, 949-970.
8: Pennybaker, A., Pezoa, S. A., & Alfano, A. (2021). Recent Advances of Chemical Definition of Cell Culture Media and Excipients for Virus and Viral Vector Manufacturing: A Review. Journal of bioprocessing & biotechniques, 11, 1-4.
9: Eagle, H. (1959). Amino acid metabolism in mammalian cell cultures. Science, 130 (3373), 432-437. https://doi.org/10.1126/science.130.3373.432
10: Fürst, P. (2000) Jonathan E. Rhoads lecture: a thirty year odyssey in nitrogen metabolism: from ammonium to dipeptides. J. Parenter. Enteral Nutr. 24, 197-209
11: Roth, E., Ollenschlager, G., Hamilton, A-, Langer, K., Fekl, W. and Jakesz, R. (1988). Influence of two glutamine containing dipeptides on growth of mammalian cells. In Vitro Cellular and Developmental Biology 24 (7), 696-698
12: Drauz, K., Knaup, G., Groeger, F. (1992). New N-acyl-peptide(s) with good solubility and stability. Patent application DE4022267A1
13: Gibco Protocol Pub No. 0007831 Rev. 1.0 FreeStyle™ F17 Expression medium accessed via https://www.thermofisher.com/document-connect/document-connect.html?url=https%3A%2F%2Fassets.thermofisher.com%2FTFS-Assets%2FLSG%2Fmanuals%2FFreeStyle_F17Expression_Medium_man.pdf (2022-03-02)
14: FUJIFILM Irvine Scientific Product User Guide BalanCD HEK293 Media System accessed via https://www.irvinesci.com/media/IrvineScientific/Resources/4/1/41084_balancd_hek293_media_sy stem.pdf (2022 Mar. 2)
15: Christie, A., & Butler, M. (1994). Glutamine-based dipeptides are utilized in mammalian cell culture by extracellular hydrolysis catalyzed by a specific peptidase. Journal of biotechnology, 37(3), 277-290. https://doi.org/10.1016/0168-1656(94)90134-1
16: Barrett, S., & Scott, J. (2011) Cell culture medium comprising small peptides, Life Technologies Corp. Patent application WO2011133902A2
17: Girard, V., Truran, R., Tuyen, O, (2019). AAV compositions, methods of making and methods of use WO2019195729A1
18: Tustian, A. D., & Bak, H. (2021). Assessment of quality attributes for adeno-associated viral vectors. Biotechnology and bioengineering, 118 (11), 4186-4203. https://doi.org/10.1002/bit.27905
19: EMA. (2018). Guideline on the quality, non-clinical and clinical aspects of gene therapy medicinal products (EMA/CAT/80183/2014). European Medicines Agency https://www.ema.europa.eu/en/documents/scientific-guideline/guideline-quality-non-clinical-clinical-aspects-gene-therapy-medicinal-products_en.pdf
20: US FDA, Center for Biologics Evaluation and Research. (2020). Chemistry, manufacturing, control (CMC) information for human gene therapy investigational new drug applications (INDs).
https://www.fda.gov/media/113760/download
21: Gao, K., Li, M., Zhong, L., Su, Q., Li, J., Li, S., He, R., Zhang, Y., Hendricks, G., Wang, J., & Gao, G. (2014). Empty Virions In AAV8 Vector Preparations Reduce Transduction Efficiency And May Cause Total Viral Particle Dose-Limiting Side-Effects. Molecular therapy. Methods & clinical development, 1(9), 20139. https://doi.org/10.1038/mtm.2013.9
22: Pei, X., Earley, L. F., He, Y., Chen, X., Hall, N. E., Samulski, R. J., & Li, C. (2018). Efficient Capsid Antigen Presentation From Adeno-Associated Virus Empty Virions In Vivo. Frontiers in immunology, 9, 844. https://doi.org/10.3389/fimmu.2018.00844
23: Qu, W., Wang, M., Wu, Y., & Xu, R. (2015). Scalable downstream strategies for purification of recombinant adeno-associated virus vectors in light of the properties. Current pharmaceutical biotechnology, 16 (8), 684-695. https://doi.org/10.2174/1389201016666150505122228
24: Cell Culture Technology for Pharmaceutical and Cell-Based Therapies, Öztürk and Wei-Shou Hu eds., Taylor and Francis Group 2006
25: Daussy, C. F., Pied, N., & Wodrich, H. (2021). Understanding Post Entry Sorting of Adenovirus Capsids; A Chance to Change Vaccine Vector Properties. Viruses, 13 (7), 1221. https://doi.org/10.3390/v13071221
26: Wu, Z., Asokan, A., & Samulski, R. J. (2006). Adeno-associated virus serotypes: vector toolkit for human gene therapy. Molecular therapy, 14(3), 316-327.
https://doi.org/10.1016/j.ymthe.2006.05.009
27: Werle, A. K., Powers, T. W., Zobel, J. F., Wappelhorst, C. N., Jarrold, M. F., Lyktey, N. A., Sloan, C. D. K., Wolf, A. J., Adams-Hall, S., Baldus, P., & Runnels, H. A. (2021). Comparison of analytical techniques to quantitate the capsid content of adeno-associated viral vectors. Molecular Therapy-Methods & Clinical Development, 23, 254-262.
https://doi.org/https://doi.org/10.1016/j.omtm.2021.08.009

### EXAMPLES

### Example 1

HEK293 cells (HEK Expi293F suspension cell line, Thermo Fisher Scientific) were employed to study the influence of glutamine-dipeptides in comparison to L-glutamine on viral vector (“DNA-containing virus particle”) production. The virus employed was adeno-associated virus 8 (AAV8).

### Materials and Methods

A lab study to evaluate the influence of glutamine-dipeptides on viral vector production (AAV8) was conducted. HEK293 cells (HEK Expi293F cells, Thermo Fisher Scientific) were pre-cultured in shake flasks in a commercial culture medium recommended for viral vector production (Freestyle F17 Expression medium, Gibco, Thermo Fisher Scientific). After thawing the cells in 20 ml of the respective media variant (final concentration of glutamine or glutamine dipeptide in media variants was 8 mM) in 125 mL shake flasks, they were cultivated for five passages at 37° C. (185 rpm/50 mm orbit, 5% CO₂) following a passaging rhythm of 3-4 days. For production, cells were split into three replicates each with a seeding viable cell density of 2×10⁶ cells/mL (day 0) in 22 mL of the respective media variant. They were transfected with polyethylenimine (PEI MAX, Polysciences) with a 2-plasmid system for AAV8 (Plasmid Factory) featuring green fluorescent protein (GFP) as gene of interest 24 hours after inoculation (day 1). Again, media variants were supplemented by glutamine (Gln), alanyl-glutamine (Ala-Gln, via the liquid supplement, GlutaMAX™, Gibco, Thermo Fisher Scientific) or glycyl-glutamine hydrate (Gly-Gln, cQrex® GQ, Evonik Operations) to a final concentration glutamine or glutamine dipeptide of 8 mM (each n=3).

To analyze cell culture performance in the different media supplementations viable cell density and viability were determined via automated cell counter. Ammonium (fluorometric approach) as well as glucose and lactate concentration (enzymatic-amperometric method) were determined during preculture and AAV8 production phase on samples from one shake flask per supplement. Transfection efficiency was determined via GFP expression by flow cytometry 48, 72 and 96 h post-transfection. For AAV8 titer determination, samples of all replicates (supernatants only) were taken 96 and 120 h post-transfection and analyzed via qPCR (genomic titer) and ELISA (capsid titer).

### Results

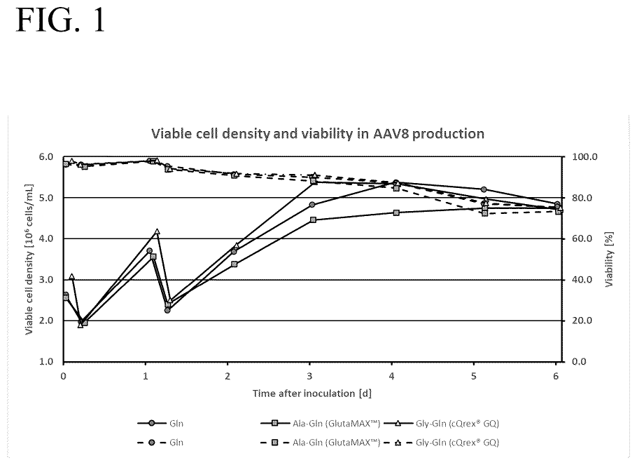

The viable cell densities and viabilities during the AAV8 production phase were highly comparable between replicates (FIG. 1). For all cultures, the viable cell density during the AAV production was in a range from ˜2.0×10⁶ cells/mL at day 0, 3.6-4.2×10⁶ cells at transfection carried out in triplicates (day 1) to a mean of 4.5×10⁶-5.4×10⁶ cells/mL per supplement at days 3 to 6. Viabilities started above 98% at the day of transfection and decreased to approx. 75% on the final day of the production period (day 6).

During AAV8 production phase, glucose concentration and lactate concentration (data not shown) were determined. Independent of the added glutamine compound, they featured similar trajectories. Glucose levels peaked with addition of fresh culture medium during transfection and declined to 0 mg/mL at day four, lactate levels stayed low throughout the process (all <2 mg/ml).

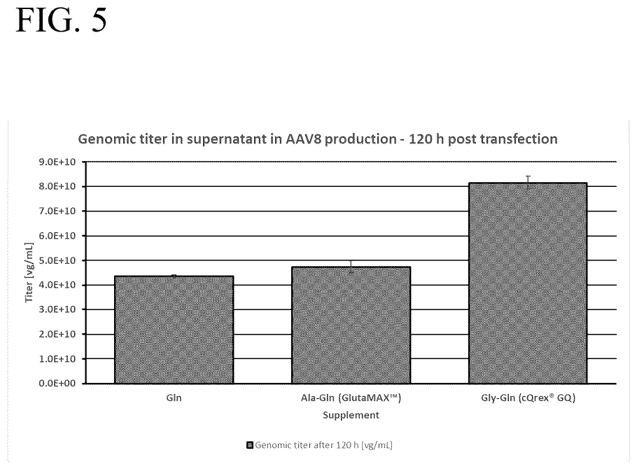

To evaluate transfection efficiency, all cultures were analyzed by flow cytometry 48 h, 72 h and 96 h after transfection (FIG. 5). Transfection efficiencies of cells supplemented with Gln, Ala-Gln and Gly-Gln varied between 78% and 86% at 48 h post transfection, between 95% and 97% at 72 h post transfection and all reached 98% at 96 h post transfection. The highest transfection efficiency was obtained with Gly-Gln. Independent of media supplement, efficiencies increased over time. In case of the measurement of transfection efficiency by fluorescence measurement of GFP-positive cells (FIG. 6), the relative fluorescence of all samples was highest 48 h post transfection with 520-572 RFU and decreased over time. At all sampling points, the culture supplemented with Gly-Gln exhibited the highest relative fluorescence.

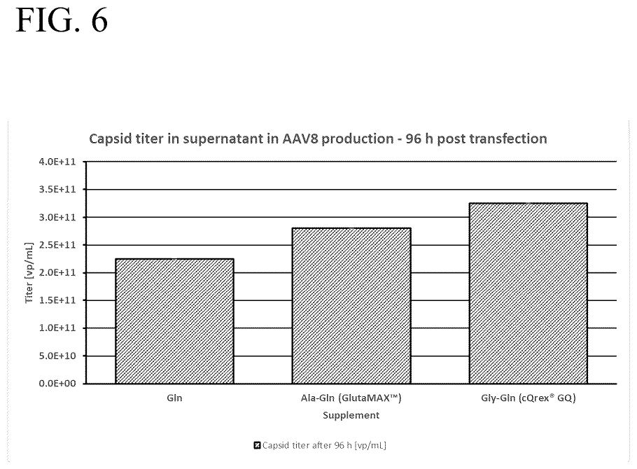

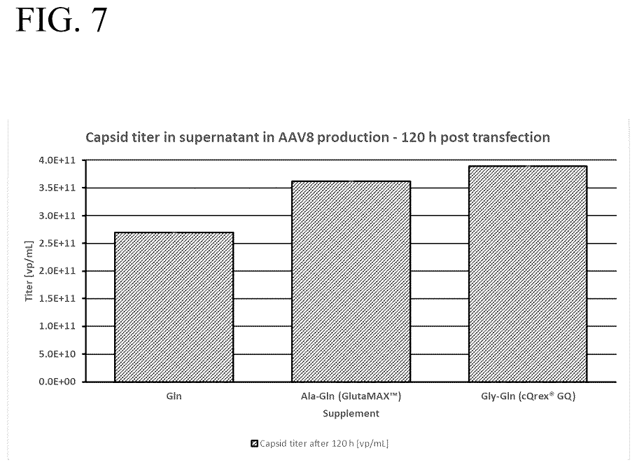

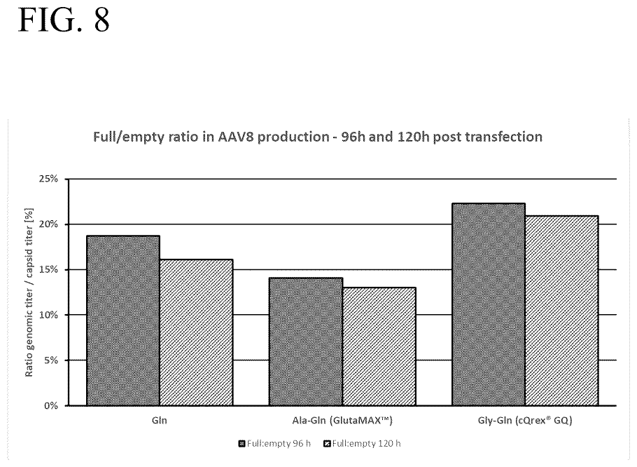

The genomic AAV8-titers of cultures supplemented with Gln, Ala-Gln and Gly-Gln were determined from supernatant at 96 h post transfection (FIG. 7) and 120 h post transfection (FIG. 8). They were in a range of 4.0-7.3×10¹⁰ vg/mL at 96 h post transfection and in a range of 4.4-8.2×10¹⁰ vg/mL at 120 h post transfection. Surprisingly, differences between the cultures could be observed, that were not expected based on similar extracellular glucose and lactate levels during production. Cultures supplemented with Gly-Gln exhibited a significantly higher genomic titer at both time points (7.3×10¹⁰ vg/mL at 96 h post transfection; 8.2×10¹⁰ vg/mL at 120 h post transfection) compared to those supplemented with Gln or Ala-Gln.

The capsid titers were determined from supernatant at 96 h post transfection (FIG. 9) and 120 h post transfection (FIG. 10). They were in a range of 2.3-3.3×10¹¹ vp/mL at 96 h post transfection and in a range of 2.7-3.9×10¹¹ vp/mL at 120 h post transfection. Compared to cultures supplemented with Gln, higher capsid titers were measured for Ala-Gln and Gly-Gln supplemented cultures.

The full/empty ratio (ratio genomic titer in vg/mL vs. capsid titer in vp/mL) was calculated using the mean values for both sampling points (FIG. 11). The full/empty ratio varied between 14-22% for the 96 h post transfection time point and between 13-21% for the 120 h post transfection time point. A small decrease between the full/empty ratio after 96 h to the full/empty ratio after 120 h could be observed in all cultures. The full/empty ratio of the cultures supplemented with Gly-Gln was significantly higher at both time points (22% at 96 h and 21% at 120 h).

FIG. 1 shows the viable cell density and viability of Expi293FT cells in FreeStyle™ F17 expression medium during AAV8 production (mean of n=3) in relation to the supplemented glutamine compound. Day 0 is the day of inoculation, day 1 is the day of transfection and day 2 to 6 correspond to sampling points 24 to 120 h post transfection.

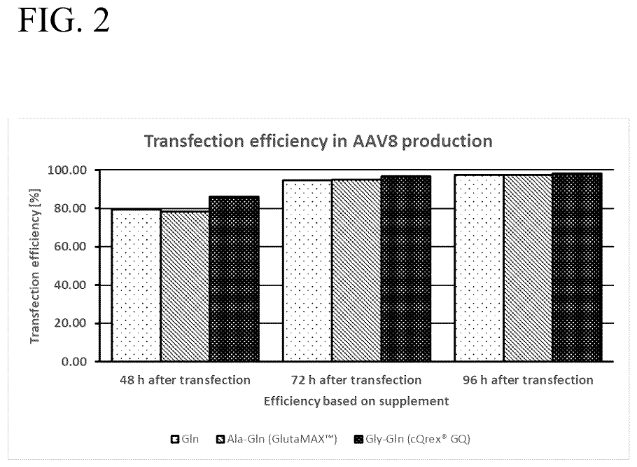

FIG. 2 shows the influence of addition of supplements in viral vector production (AAV8) using HEK Expi293F cells in Freestyle F17 Expression medium at 48, 72 and 96 hours after transfection. Plot of the transfection efficiency in relation to the supplemented glutamine compound.

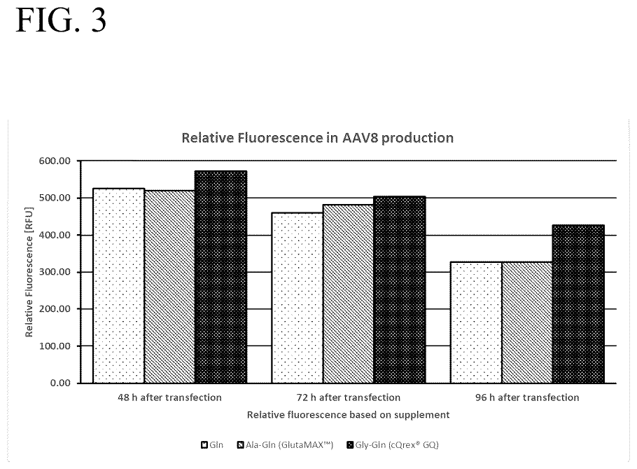

FIG. 3 shows the influence of addition of supplements in viral vector production (AAV8) using HEK Expi293F cells in Freestyle F17 Expression medium at 48, 72 and 96 hours after transfection. Plot of the relative fluorescence in relation to the supplemented glutamine compound.

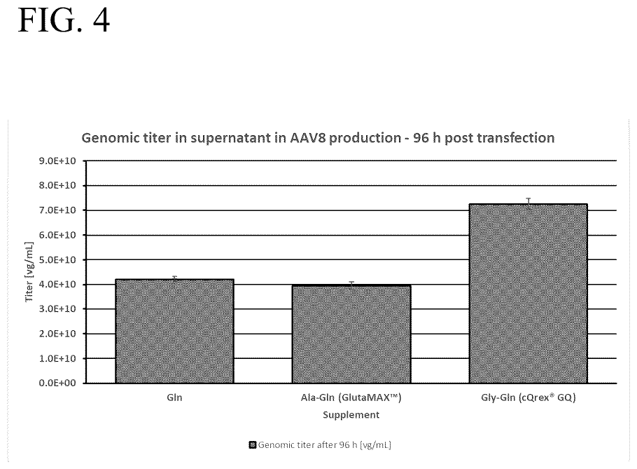

FIG. 4 shows the influence of addition of supplements in viral vector production (AAV8) using HEK Expi293F cells in Freestyle F17 Expression medium at 96 hours after transfection. Plot of the viral genome titer (mean of n=3±SD) in relation to the supplemented glutamine compound.

FIG. 5 shows the influence of addition of supplements in viral vector production (AAV8) using HEK Expi293F cells in Freestyle F17 Expression medium at 120 hours after transfection. Plot of the viral genome titer (mean of n=3±SD) in relation to the supplemented glutamine compound.

FIG. 6 shows the influence of addition of supplements in viral vector production (AAV8) using HEK Expi293F cells in Freestyle F17 Expression medium at 96 hours after transfection. Plot of the capsid titer (mean of n=3±SD) in relation to the supplemented glutamine compound.

FIG. 7 shows the influence of addition of supplements in viral vector production (AAV8) using HEK Expi293F cells in Freestyle F17 Expression medium at 120 hours after transfection. Plot of the capsid titer (mean of n=3±SD) in relation to the supplemented glutamine compound.

FIG. 8 shows the influence of addition of supplements in viral vector production (AAV8) using HEK Expi293F cells in Freestyle F17 Expression medium at 96 and 120 hours after transfection. Plot of the full/empty ratio (ratio viral genome titer vs. viral particle titer) in relation to the supplemented glutamine compound.

### CONCLUSION

The AAV8 production phase was monitored for 120 h post transfection. The evaluation of transfection efficiency by fluorescence measurement of green-fluorescent protein-positive cells, the capsid titer in the supernatant determined by ELISA as well as the genomic titer determined from supernatant by qPCR surprisingly indicated comparably high to slightly more efficient transfection as well as increased genomic and capsid titer for the cultures supplemented with Gly-Gln compared to the other variants. The increase of genomic titer was more pronounced than the increase of capsid titer, which is reflected in the highest full/empty ratios for cultures supplemented with Gly-Gln. Compared to Ala-Gln supplementation, which resulted in a higher capsid titer while featuring genomic titers comparable to the other processes, the high genomic titer is remarkable indicating more efficient production and addressing the challenges related to empty capsids and the full/empty-ratio described above.

1. A method of manufacturing an RNA- or DNA-containing virus particle in cell culture, the method comprising:
providing a cell capable of producing said virus particle;
contacting said cell with a culture medium comprising one or more dipeptides, wherein one dipeptide is glycyl-glutamine (Gly-Gln); and
obtaining said RNA- or DNA-containing virus particle from said culture medium or from said cell.

2. The method according to claim 1, wherein a full/empty ratio is determined by division of genome titer by PCR and capsid titer by ELISA.

3. The method according to claim 1, wherein the virus particles are particle is at least one selected from the group consisting of Adenoviruses, Adeno-associated viruses (AAV), and lentiviruses.

4. The method according to claim 1, wherein the dipeptide is present in the culture medium at a concentration of from 0.1 mM to 20 mM.

5. The method according to claim 1, wherein the culture medium further comprises:
at least one carbohydrate,
at least one free amino acid,
at least one inorganic salt,
a buffering agent, and/or
at least one vitamin.

6. The method according to claim 1, wherein the cell is selected from the group consisting of CHO cells, COS cells, VERO cells, BHK cells, HEK cells, HELA cells, AE-1 cells, insect cells, Sf9 cells, TT-D6 cells, BLKCL.4 primary skin fibroblasts, A549 human adenocarcinoma cells, MDCK cells or enders cells, or fibroblast cells, muscle cells, nerve cells, stem cells, skin cells, endothelial cells, immune cells, and hybridoma cells.

7. (canceled)

8. The method according to claim 3, wherein the virus particle is selected from the group consisting of Adeno-associated viruses AAV8 and AAV2.

9. The method according to claim 4, wherein the dipeptide is present in the culture medium at a concentration of from 5 mM to 10 mM.

10. The method according to claim 6, wherein the cell is selected from the group consisting of NK cells, T-cells and HEK cells.
